(19) United States
(12) Patent Application Publication
(10) Pub. No.: US 2026/0345851 A1
(43) Pub. Date: Nov. 5, 2026

(54) AROMA DIFFUSER

(71) Applicant: AROMA PERFECTO LLC, Diamond Bar, CA (US)

(72) Inventor: Yong WU, Guangzhou (CN)

(73) Assignee: AROMA PERFECTO LLC, Diamond Bar, CA (US)

(21) Appl. No.: 19/663,689

(22) Filed: Apr. 30, 2026

(30) Foreign Application Priority Data
Apr. 30, 2025 (CN) 202520863297.8

(51) Int. Cl. A61L 9/14; F24F 8/50

(52) U.S. Cl. CPC A61L 9/14; F24F 8/50; A61L 2209/134

(57) ABSTRACT

An aroma diffuser includes: a main body; an atomization head in sealing connection with the main body via a sealing element; a solution container coupled to the atomization head. A pressure chamber is formed between the solution container and the main body; and a high-pressure air generator, configured to supply pressurized air to the atomization head for atomization through the pressure chamber. The atomization head is configured to draw the solution from the solution container and mix the solution with the pressurized air to form a mist, and the mist is released from the atomization head. A buffer chamber is further provided, and the pressurized air is supplied from the high-pressure air generator to the pressure chamber through the buffer chamber.

### CROSS-REFERENCE TO RELATED APPLICATIONS

The present disclosure claims priority to Chinese Application No. 202520863297.8, filed on Apr. 30, 2025, the content of which is incorporated herein by reference in its entirety.

### TECHNICAL FIELD

The present disclosure relates to the field of atomization devices, and more particularly, to an aroma diffuser.

### BACKGROUND

Aroma diffusers are widely used household appliances that convert aroma solution into fine mist for aromatherapy, humidification, and air purification. Among these, high-pressure air atomization diffusers are particularly popular due to their high efficiency and rapid aroma dispersion.

In the high-pressure air atomization diffusers, aroma liquid is suctioned, due to negative pressure caused by high-pressure air, into a mixing chamber, where the liquid and high-speed air are atomized into fine particles, and finally released as a mist to create a pleasant aromatic atmosphere.

In the present high-pressure air atomization diffuser, a high-pressure air generator, such as an air pump, is usually used to provide high-pressure air which is delivered to a pressure chamber of the diffuser. In order to further supply the high-pressure air from the pressure chamber to the atomization assembly, the atomization assembly is formed with an air inlet which is positioned within the pressure chamber. The advantage of such configuration is that once the atomization assembly is fastened to the main body, a sealed pressure chamber is formed via a sealing assembly, and the air inlet is right inside the pressure chamber, regardless of assembly orientation or angle. This structure simplifies assembly and eliminates concerns regarding air leakage. Therefore, the pressure chamber plays a critical role, which receives high-pressure air from the air pump and guides it to the atomization head, providing a stable air supplying with sufficient pressure, thereby ensuring that the aroma liquid can be successfully atomized into a uniform mist.

However, in actual applications, the present design of the pressure chamber suffers from several issues.

On one hand, the pressure within the pressure chamber is difficult to control. Variations in the shape and dimensions of the pressure chamber may alter the flow path and spatial distribution of the air within the chamber, making it difficult to establish a desired stable pressure environment when the high-pressure air from the air pump enters the chamber. In addition, when the shape of the pressure chamber is relatively complex or the dimensions of the pressure chamber are out of tolerance, irregular flow phenomena such as eddy currents or backflow may occur within the chamber. This leads to a significant deviation between the actual pressure acting on the atomization head and the set output pressure of the air pump. Such pressure instability results in inconsistent atomization effects, where the volume and uniformity of the mist cannot be effectively guaranteed, thereby affecting the overall performance and use experience of the aroma diffuser.

On the other hand, the pressure distribution within the pressure chamber is uneven. In cases where the size specifications of the pressure chamber are not uniform, there are significant pressure differences at different positions within the chamber. Particularly, the air pressure close to the air pump inlet or certain specific regions of the pressure chamber may be relatively high, while the pressure in regions further away may be lower. This uneven pressure distribution results in inconsistent pneumatic action on different parts of the atomization head, causing variations in the degree of atomization of the aroma liquid across the atomization head.

### SUMMARY

The present disclosure therefore provides an improved aroma diffuser, which may allow for easy control of the pressure within the pressure chamber and ensure uniform pressure distribution therein.

The aroma diffuser according to the present disclosure includes a main body, an atomization head, and a solution container for containing a solution to be atomized. The atomization head is coupled to the solution container, and configured to draw the solution from the solution container and mix it with pressurized air to form a mist, which is finally released from the atomization head. A sealing element is further arranged between the atomization head and the main body to achieve a sealed connection therebetween. A pressure chamber is formed between the solution container and the main body. A high-pressure air generator is further included to supply the pressurized air to the pressure chamber, and the pressurized air is further delivered from the pressure chamber to the atomization head. In the aroma diffuser of the present disclosure, a buffer chamber is further included. In such configuration, the pressurized air is supplied to the pressure chamber through the buffer chamber.

The buffer chamber in the present disclosure can reduce the flow rate of the pressurized air, leading to a more uniform pressure distribution and an improved pressure stability inside the pressure chamber. Furthermore, this can further effectively absorb and disperse the noise generated by the pressurized air, thereby reducing the operation noise of the diffuser. The buffer chamber also prevents the aroma liquid from entering the high-pressure air generator, which enhances the use experience.

In a particular embodiment of the present disclosure, the main body may particularly include an inner cavity for containing the solution container and a housing for containing the inner cavity. The pressure chamber may be formed between the solution container and the inner cavity. The sealing element may be positioned at the upper rim of the inner cavity to seal against the atomization assembly. Furthermore, the high‑pressure air generator may be in communication with the buffer chamber via an air supply pipe, such that the pressurized air may be supplied from the high-pressure air generator to the buffer chamber and further directed to the pressure chamber. Preferably, the high-pressure air generator may be positioned between the inner cavity and the housing, especially at the bottom of the inner cavity, which may be in communication with the inner cavity via an upwardly extending air supply pipe.

In this configuration, the inner cavity and the housing are designed in a layered structure, and the container, sealing assembly, pressure chamber, and high-pressure air generator are rationally arranged, rendering the overall structure of the diffuser more compact and efficient.

In a further advantageous embodiment of the present disclosure, the buffer chamber may be formed surrounding the inner cavity, with an inlet in communication with the air supply pipe and a plurality of outlets in communication with the inner cavity. The circumferentially arranged buffer chamber provides superior structural support, enhancing overall stability. In addition, the provision of multiple air outlets allows the high-pressure air to enter the inner cavity more uniformly.

More particularly, the multiple air outlets may be preferably slit-shaped, extending vertically downward from the upper portion of the inner cavity, with the lower ends of the air outlets maintaining a certain distance from the underside of the buffer chamber. The slit-shaped air outlets may maintain the requisite outlet size to mitigate local pressure fluctuations. Furthermore, as oversized openings typically compromise the structural integrity of the buffer chamber, the slit-shaped air outlets contribute to improved structural strength. In addition, the slit-shaped air outlets are positioned at a predetermined distance from the bottom of the buffer chamber, thereby defining a sump region at the bottom of the buffer chamber. Accordingly, any atomized aroma liquid entering the buffer chamber can be condensed and accumulated within this region, rather than flowing into the air supply pipe. This configuration ensures reliable operation of the diffuser.

Preferably, the sealing element may particularly include an outer sealing ring engaged around an outer side of the upper rim of the inner cavity, an inner sealing ring engaged around an inner side of the upper rim of the inner cavity, and a connection portion integrally connecting an upper end of the outer sealing ring to an upper end of the inner sealing ring. The double sealing ring configuration can offer dual protection, considerably improving the sealing of the inner cavity. Furthermore, the connection portion in the present disclosure serves as a mounting and positioning datum, which may streamline the assembly procedure.

To better secure the sealing assembly and prevent displacement-induced sealing degradation during repeated pressing by the atomization head, the connection portion may extend outwardly beyond the outer sealing ring to define a compression portion. This design enables the compression portion to be simultaneously pressed and fixed by other internal structures of the main body when the sealing assembly is installed onto the upper rim of the inner cavity. More particularly, the compression portion may be clamped by the main body. In addition, a friction surface may be provided at least of the main body and the compression portion to resist relative movement.

In a more preferable embodiment of the present disclosure, in order to enable smooth engagement between the sealing element and the atomization head, the connection portion may be further formed with a transitional curved surface extending toward the inner sealing ring. Moreover, the outer sealing ring may project beyond the bottom end of the inner sealing ring, allowing the inner sealing ring to deform downward slightly under compression of the atomization head to provide continuous elastic sealing. This may further improve sealing performance.

For the atomization head of the aroma diffuser, to ensure uniform compression of the sealing element, the atomization head may be formed with a pressing shoulder corresponding to the inner sealing ring, which pressed axially against the inner sealing ring.

Additionally or alternatively, at least the lower portion of the atomization head may in form of a tapered structure that abutted against the inner sealing ring, causing the inner sealing ring to gradually deform along the taper.

In an alternative embodiment, the inner sealing ring may be inwardly formed with a stepped portion, which extends downward to form one or more steps. Consequently, when the tapered structure presses down, the stepped portion may flex to accommodate deformation, while ensuring a tight seal.

Therefore, the diffuser of the present disclosure may eliminate the need for complex fine-tuning of the controlling of the high-pressure air generator for different models during production. This may significantly reduce manufacturing difficulty and costs, while improving production efficiency and product consistency. Consequently, the disclosed diffuser may offer a more stable and efficient performance, contributing to the technological advancement and healthy development of the aroma diffuser industry.

The above and other features of the present disclosure, including various novel details of construction and combinations of parts, will now be more particularly described with reference to the accompanying drawings and pointed out in the claims. It will be understood that the particular methods and apparatus embodying the disclosure are shown by way of illustration and not as a limitation of the disclosure. As will be readily apparent to those skilled in the art, various changes and modifications may be made without departing from the spirit and scope of the disclosure.

### BRIEF DESCRIPTION OF THE DRAWINGS

FIG. 1 is a perspective view of an aroma diffuser according to an embodiment of the present disclosure.

FIG. 2 is an exploded view of the aroma diffuser in FIG. 1.

FIG. 3 is a front view of the aroma diffuser in FIG. 1.

FIG. 4 is a cross-sectional view of the aroma diffuser taken along line A–A of FIG. 3.

FIG. 5 is a partial assembly view of the aroma diffuser in FIG. 1, with some parts omitted.

FIG. 6 is an enlarged detail showing the assembly of the main body 100 and an atomization head 200, with some parts omitted.

FIG. 7 is a perspective view of a main body of the aroma diffuser in FIG. 1.

FIG. 8 is an enlarged detail view of portion I in FIG. 7.

FIG. 9 is a perspective section view of the main body of the aroma diffuser along line A–A in FIG. 3.

FIG. 10 is an enlarged detail view of portion II in FIG. 9.

FIG. 11 is another perspective view of the main body of the aroma diffuser in FIG. 1.

FIG. 12 is an enlarged detail view of portion III in FIG. 11.

FIG. 13 is a structural view showing a buffer chamber.

FIG. 14 is a perspective section view of a sealing element according to an embodiment of the present disclosure.

FIG. 15 is a perspective section view of the aroma diffuser taken along line A–A of FIG. 3, with some parts omitted.

FIG. 16 is an enlarged detail view of portion IV in FIG. 15.

FIG. 17 is another perspective view of the main body of the aroma diffuser in FIG. 1.

FIG. 18 is a cross‑sectional view of an aroma diffuser according to another embodiment of the present disclosure.

FIG. 19 is a perspective section view of the main body in FIG. 18.

FIG. 20 is an enlarged detail view of portion V in FIG. 19.

FIG. 21 is a perspective section view of a sealing element of according to another embodiment of the present disclosure.

FIG. 22 is a top view of the aroma diffuser in FIG. 18.

FIG. 23 is a perspective section view taken along line B–B of FIG. 22.

FIG. 24 is an enlarged detail view of portion VI in FIG. 23.

### DETAILED DESCRIPTION OF THE EMBODIMENTS

The drawings are merely for illustrative purposes and should not be interpreted as limitations to this patent. To better illustrate the embodiments, some components in the drawings may be omitted, enlarged, or reduced, and do not represent the actual size of the product. For those skilled in the art, it can be understood that some well-known structures and their descriptions may be omitted in the drawings. The positional relationships depicted in the drawings are for illustrative purposes only and are not to be construed as limiting this patent.

Reference will now be made in detail to embodiments of the present disclosure, examples of which are illustrated in the accompanying drawings.

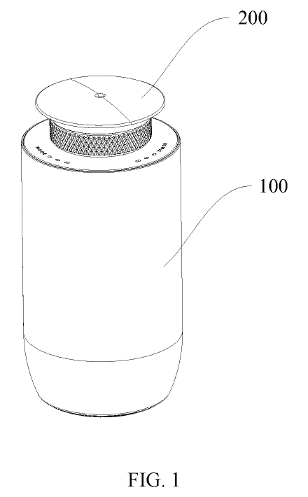

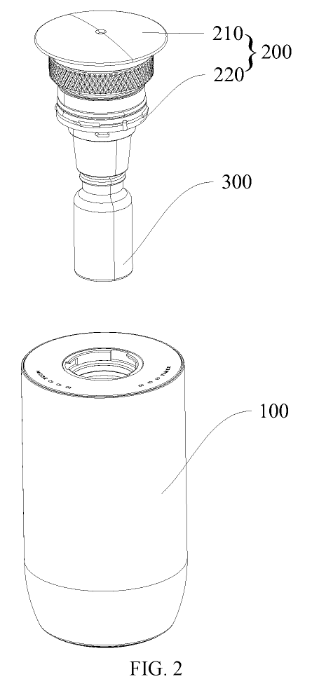

FIG. 1 provides an aroma diffuser according to an embodiment of the present disclosure, including a main body 100 and an atomization head 200. Referring to FIG. 2, the atomization head 200 particularly includes a cover 210 and an atomization assembly 220. The atomization assembly 220 is coupled to a solution container 300 for containing the solution to be atomized. The atomization assembly 220 is configured to draw the solution from the solution container 300 and mix it with the pressurized air to form a mist, which is finally released from the cover 210.

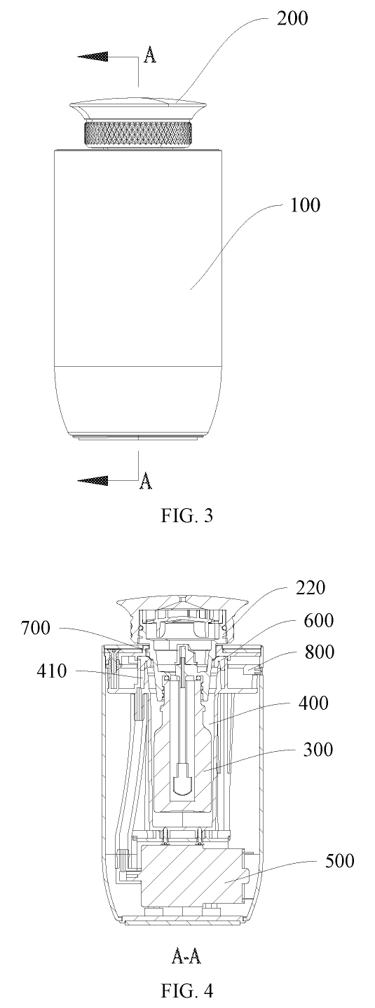

In combination with FIGS. 3 and 4, the atomization head 200 is in sealing connection with the main body 100, especially through a sealing element 600. A pressure chamber 400 is formed between the solution container 300 and the main body 100. A high‑pressure air generator 500 for supplying pressurized air to the pressure chamber 400 is further arranged at the bottom of the main body 100. The pressurized air is finally fed to the atomization assembly 220 from the pressure chamber 400. A control module 800 is further provided within the main body 100.

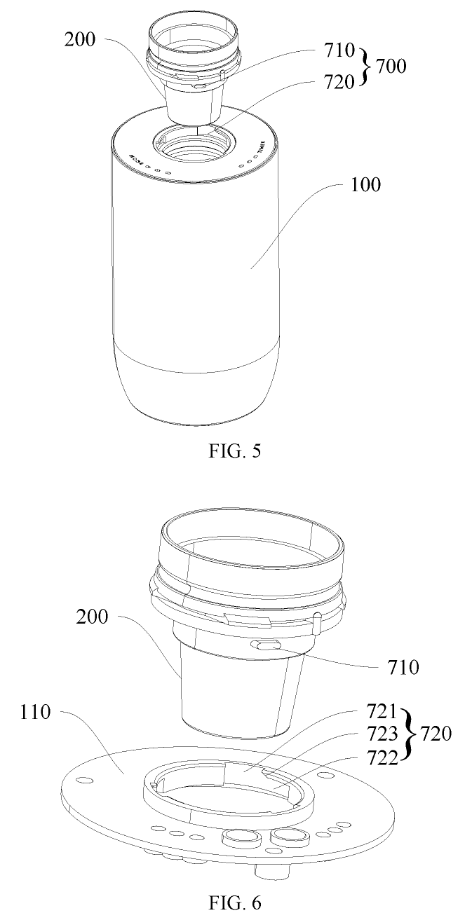

In combination with FIGS. 2 and 5, the atomization head 200 is coupled to the main body 100 by three circumferentially uniformly distributed snap‑fit elements 700 in this embodiment. Each snap‑fit element 700 includes a snap lug 710 protruding from the atomization head 200, and a snap groove 720, which substantially has an L-shaped cross section, recessed in the main body 100. Referring to FIG. 6, the snap groove 720 particularly includes a vertical section 721 allowing the snap lug 710 to be vertically inserted, and a transverse section 722 allowing the snap lug 710 to be transversely inserted, and a transitional section 723 connecting the vertical section 721 to the transverse section 722. The transitional section 723 in this embodiment has an inclined surface. In such configuration, when the snap lug 710 is snapped into the snap groove 720, the atomization head 200 and the main body 100 are pressed against the sealing element 600 to achieve sealing.

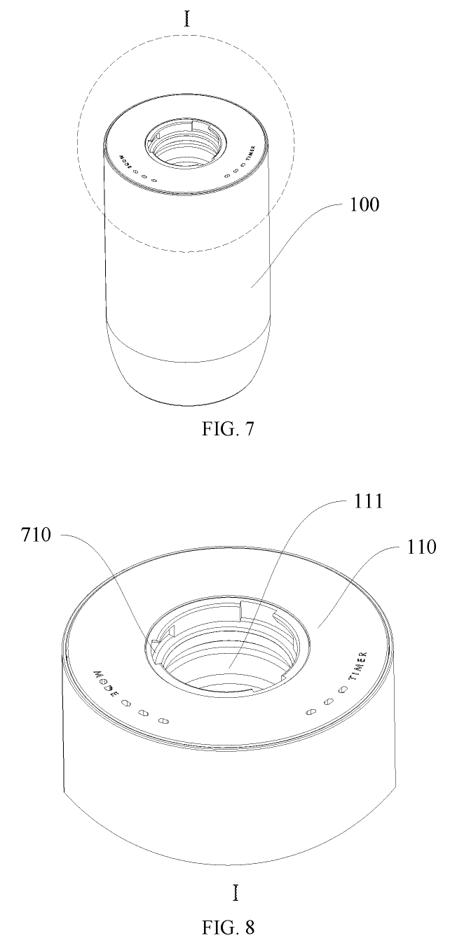

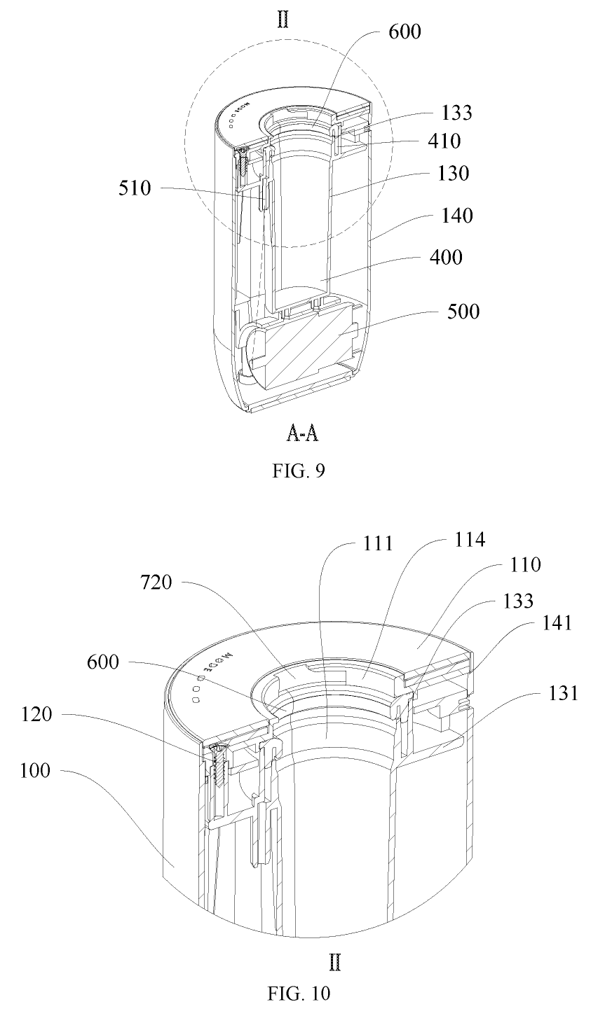

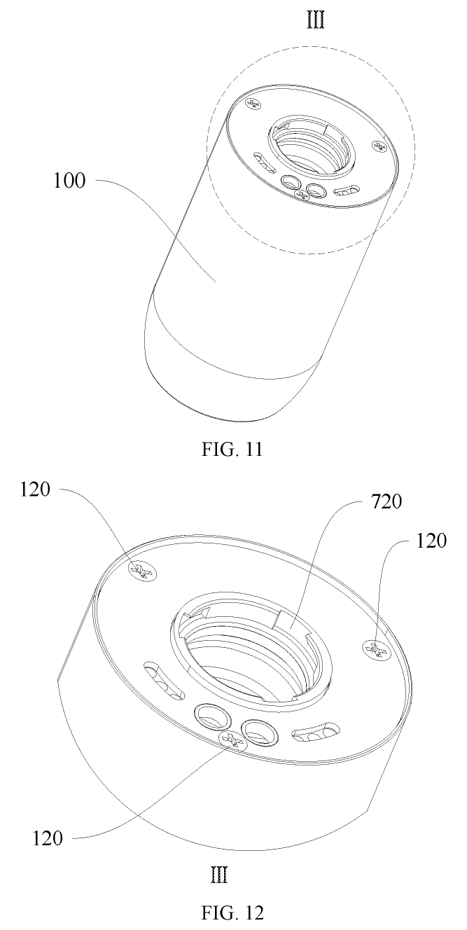

In combination with FIGS. 7 and 8, a control panel 110 is provided on the upper end of the main body 100. The control panel 110 is formed with a central opening 111 for the atomization head 200 and the solution container 300 passing therethrough. The snap grooves 720 are circumferentially formed on the inner wall of the opening 111. Further in combination with FIGS. 9 and 10, the control panel 110 is fastened to the main body 100, especially by fasteners 120. Further in combination with FIGS. 11 and 12, the fasteners 120 are arranged circumferentially around the opening 111 and corresponding to the snap‑fit elements 700.

Referring back to FIG. 9, the main body 100 in this embodiment particularly includes an inner cavity 130 and a housing 140 for containing the inner cavity 130. As shown in FIG. 4, the solution container 300 is positioned inside the inner cavity 130. The sealing element 600 is positioned at the upper rim 133 of the inner cavity 130 to seal against the atomization assembly 220. The pressure chamber 400 is particularly formed between the solution container 300 and inner cavity 130. The high‑pressure air generator 500 is disposed between the inner cavity 130 and the housing 140, which is communicated with the pressure chamber 400 via an air supply pipe 510. A buffer chamber 410 is further provided in this embodiment. In such configuration, the pressurized air from the high‑pressure air generator 500 is directed into the pressure chamber through the buffer chamber via the air supply pipe 510.

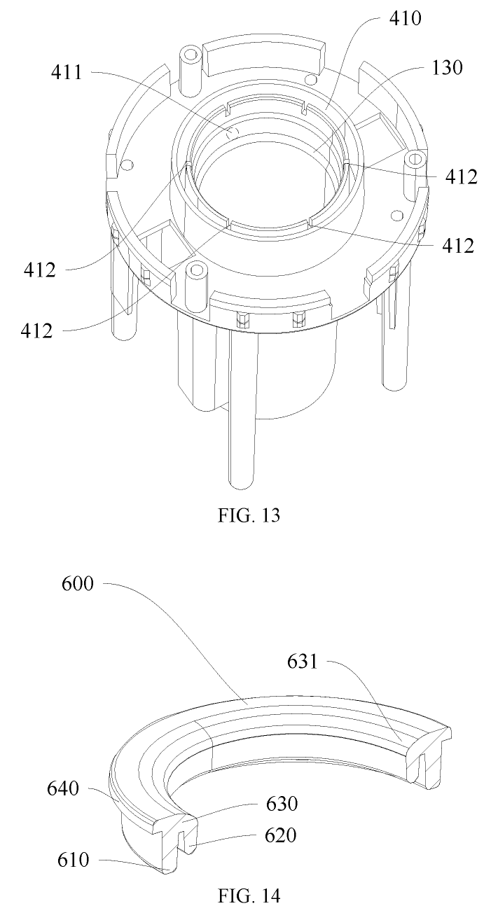

Referring now to FIG. 13, the buffer chamber 410 is formed surrounding the inner cavity 130, with an inlet 411 communicated with the air supply pipe 510 and a plurality of outlets 412 communicated with the inner cavity 130. In this embodiment, the outlets 412 are slit‑shaped, which are vertically extended down from the upper end of the inner cavity 130, especially from the upper rim of the inner cavity 130, with the lower end of each outlet 412 spaced a distance from the underside of the buffer chamber 410, namely the lower end of the outlets 412 is not flush with the underside of the buffer chamber 410.

In combination with FIGS. 10 and 14, the sealing element 600 in this embodiment particularly includes an outer sealing ring 610 engaged around the outer side of the upper rim 133 of the inner cavity 130, an inner sealing ring 620 engaged around the inner side of the upper rim 133 of the inner cavity 130, and a connection portion 630 integrally connecting the upper end of the outer sealing ring 610 to the upper end of the inner sealing ring 620. The outer sealing ring 610 projects beyond the bottom end of the inner sealing ring 620. The connection portion 630 is formed with a transitional curved surface 631 extending toward the inner sealing ring 620. Furthermore, the connection portion 630 is radially extended outward from the outer sealing ring 610 to form a compression portion 640.

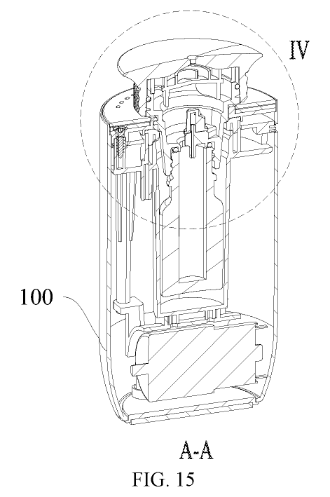

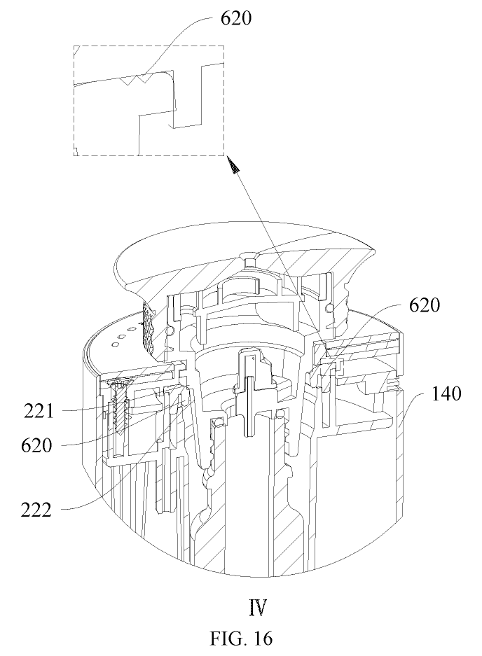

In combination with FIGS. 15 and 16, the compression portion 640 is pressed and fastened by the main body 100. A friction surface 641 is provided on the contact surface between the compression portion 640 and the main body 100 - especially on the compression portion 640 - to avoid relative movement therebetween. The atomization assembly 220 is formed with a pressing shoulder 221 corresponding to the inner sealing ring 620. The pressing shoulder 221 is pressed against the inner sealing ring 620.

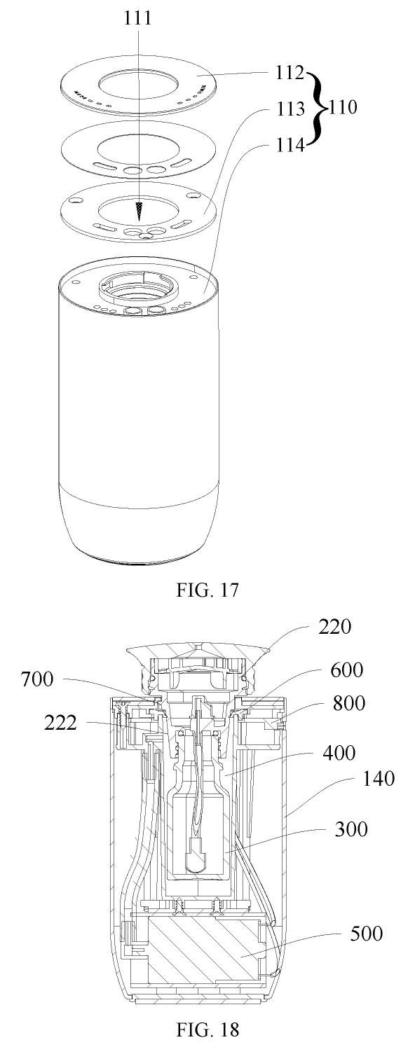

Referring to FIG. 17, the control panel 110 in this embodiment particularly includes a display layer 112, a metal support layer 113, and a holder 114 arranged from top to bottom. In conjunction with FIG. 10, the snap grooves 720 of the snap-fit elements 700 are formed at the inner side of the holder 114. Particularly, the holder 114 may be formed with a flange around the opening 111, which is perpendicular to the display layer 112 and the metal support layer 113. The snap grooves 720 may be preferably formed in the flange. The upper portion of the inner cavity 130 is extended toward the housing 140 to form a support 131. An upper portion of the housing 140 is formed with a support shoulder 141. The holder 114 is rested on the inner cavity 130 and the support shoulder 141. The sealing element 600 is fixed between the holder 114 and the inner cavity 130. In this embodiment, the inner cavity 130 is connected to the holder 114, especially the lower side of the holder 114. Accordingly, the inner cavity 130 in this embodiment is supported inside the housing through the holder 114.

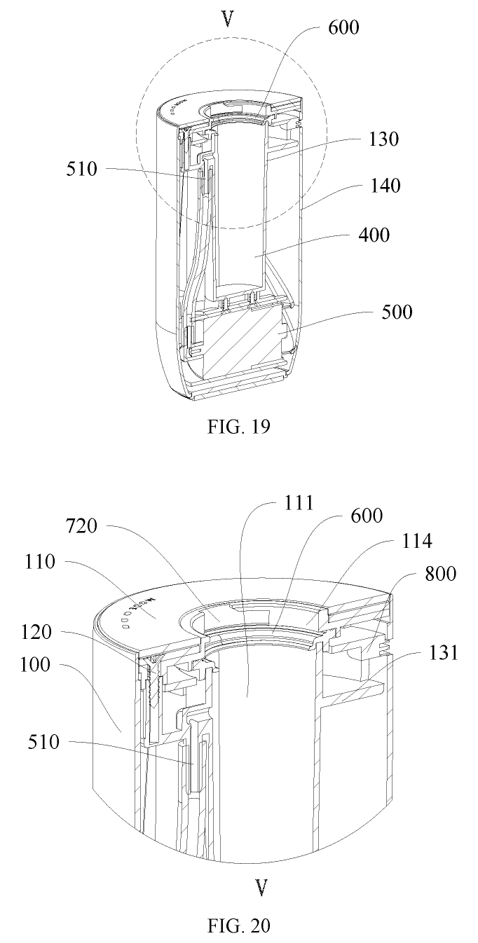

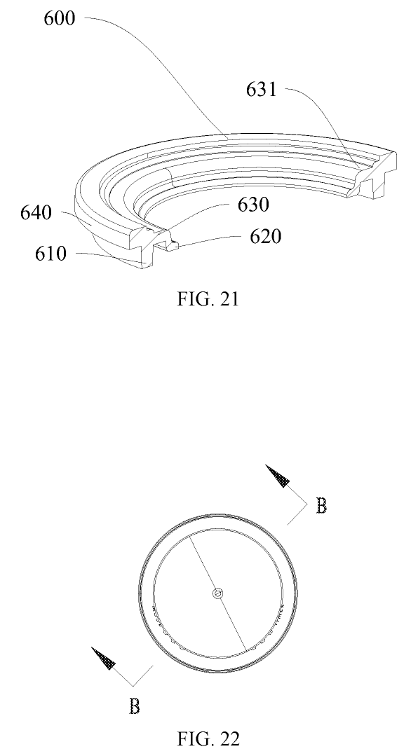

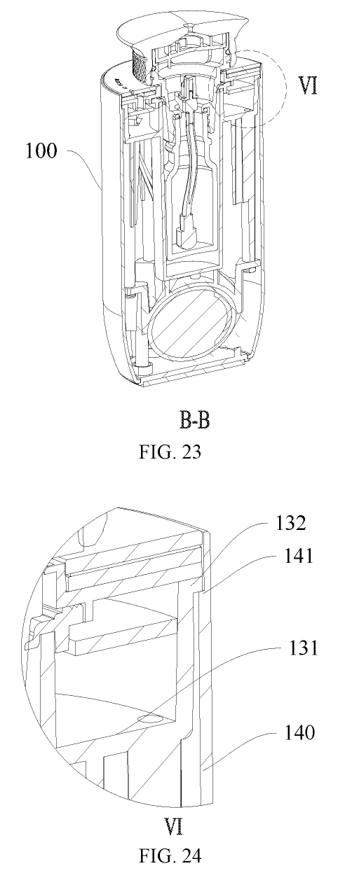

FIG. 18 shows a cross section of an aroma diffuser according to another embodiment of the present disclosure. FIG. 19 shows a cross section of the main body of the aroma diffuser in FIG. 18. In this embodiment, as clearly shown in FIG. 21, the buffer chamber 410 is not provided. That is, the air supply pipe 510 in this embodiment is directedly communicated with the pressure chamber 400 through the outlet 412.

Referring to FIG. 21, the inner sealing ring 620 of the sealing element 600 in this embodiment is further inwardly formed with a stepped portion. The lower portion of the atomization assembly 220 in this embodiment is designed as a tapered structure 222 (clearly shown in FIG. 18), which is abutted against the inner sealing ring 620, especially the stepped portion of the inner sealing ring 620.

In the present embodiment, the inner sealing ring 620 has an inner diameter smaller than the diameter of the opening 111. When the atomization assembly 220 is inserted into the opening 111 to engage with the main body 100, the tapered surface 222 progressively contacts the inner sealing ring 620 from a smaller diameter end to a larger diameter end. Upon contact, the tapered surface 222 presses the inner sealing ring 620 downwards, causing the stepped portion to deflect downwards. Due to good elastic restoring properties, the stepped portion is abutted tightly against the tapered surface 222 to achieve sealing between the atomization assembly 220 and the inner cavity 130.

Referring to FIGS. 22 -24, in this embodiment, the housing 140 is formed with a support shoulder 141 at the upper portion. Correspondingly, the support 131 of the inner cavity 130 is formed with a flange 132. The flange 132 is supported on the support shoulder 141 of the housing 140. In this embodiment, the holder 114 is supported on the support shoulder 141 of the housing 140. Accordingly, the inner cavity 130 in this embodiment is supported inside the housing through the support 131.

Apparently, the above embodiments of the present disclosure are only examples provided to clearly illustrate the present disclosure, and are not limitations on the implementations of the present disclosure. For those of ordinary skill in the art, various other forms of modifications or changes can be made on the basis of the above description. It is neither necessary nor possible to enumerate all embodiments herein. Any modifications, equivalent replacements, improvements, etc. made within the spirit and principle of the present disclosure shall be included within the scope of protection of the claims of the present disclosure.

What is claimed is:

1. An aroma diffuser, comprising:
a main body;
an atomization head, configured to form a sealed connection with the main body via a sealing element;
a solution container for containing a solution to be atomized, the solution container being coupled to the atomization head, wherein a pressure chamber is formed within the main body;
a high-pressure air generator, configured to supply pressurized air to the atomization head for atomization through the pressure chamber, wherein the atomization head is configured to draw the solution from the solution container and mix the solution with the pressurized air to form a mist, and the mist is released from the atomization head; and
a buffer chamber, wherein the pressurized air is supplied from the high-pressure air generator to the pressure chamber through the buffer chamber.

2. The aroma diffuser according to claim 1, wherein
the main body comprises an inner cavity for containing the solution container and a housing for containing the inner cavity, and the pressure chamber is formed between the solution container and the inner cavity;
the sealing element is positioned at an upper rim of the inner cavity to seal against the atomization head; and
the high‑pressure air generator is in communication with the buffer chamber via an air supply pipe, such that the pressurized air is supplied from the high-pressure air generator to the buffer chamber and further directed to the pressure chamber.

3. The aroma diffuser according to claim 2, wherein the buffer chamber is formed surrounding the inner cavity, with an inlet in communication with the air supply pipe and a plurality of outlets in communication with the inner cavity.

4. The aroma diffuser according to claim 3, wherein the plurality of outlets are slit‑shaped, and the slit-shaped outlets vertically extend down from an upper end of the inner cavity, with a lower end of each outlet spaced a distance from an underside of the buffer chamber.

5. The aroma diffuser according to claim 2, wherein the sealing element comprises:
an outer sealing ring engaged around an outer side of the upper rim of the inner cavity,
an inner sealing ring engaged around an inner side of the upper rim of the inner cavity, and
a connection portion integrally connecting an upper end of the outer sealing ring to an upper end of the inner sealing ring.

6. The aroma diffuser according to claim 5, wherein the connection portion extends radially outward from the outer sealing ring to form a compression portion.

7. The aroma diffuser according to claim 6, wherein the compression portion is clamped by the main body, and at least one of the compression portion and the main body is formed with a friction surface.

8. The aroma diffuser according to claim 5, wherein the connection portion is formed with a transitional curved surface extending toward the inner sealing ring, and the outer sealing ring projects beyond a bottom end of the inner sealing ring.

9. The aroma diffuser according to claim 5, wherein the inner sealing ring of the sealing element is formed with an inwardly extending stepped portion.

10. The aroma diffuser according to claim 5, wherein the atomization head is formed with a pressing shoulder corresponding to the inner sealing ring, and the pressing shoulder is configured to press axially against the inner sealing ring.

11. The aroma diffuser according to claim 5, wherein at least a lower portion of the atomization head is in a form of a tapered structure, and the tapered structure is configured to abut against the inner sealing ring.
